(19) United States
(12) Patent Application Publication
(10) Pub. No.: US 2026/0199232 A1
(43) Pub. Date: Jul. 16, 2026

(54) NEUROPROTECTIVE DRUG SUBSTANCE-LOADED NANOMOTOR FORMULATIONS

(71) Applicant: HACETTEPE UNIVERSITESI, Ankara (TR)

(72) Inventors: Hakan EROGLU, Ankara (TR); Filiz KURALAY, Ankara (TR); Gizem TEZEL, Ankara (TR); Elif OZTURK, Ankara (TR); Selin Seda TIMUR, Ankara (TR); Reyhan Neslihan GURSOY, Ankara (TR); Levent ONER, Ankara (TR); Kezban ULUBAYRAM, Ankara (TR)

(73) Assignee: HACETTEPE UNIVERSITESI, Ankara (TR)

(21) Appl. No.: 19/134,258

(22) PCT Filed: Dec. 18, 2023
(86) PCT No.: PCT/TR2023/051570

§ 371 Date: May 30, 2025

(30) Foreign Application Priority Data
Dec. 19, 2022 (TR) 2022/019723

(51) Int. Cl. A61K 9/00; A61K 31/13; B82Y 5/00

(52) U.S. Cl. CPC A61K 9/0097; A61K 31/13; B82Y 5/00

(57) ABSTRACT

A polymer layer containing a pharmaceutical drug substance-loaded amino group and a nanomotor containing a metal layer for orientation by a magnetic field is used to treat Alzheimer's Disease; Memantine HCl-loaded nanomotors are synthesized with a biocompatible poly-L-lysine polymer whose movement is directly controlled by the magnetic field and targets the brain by crossing the blood brain barrier by providing movement in the magnetic field.

### CROSS REFERENCE TO RELATED APPLICATIONS

This application is a national stage entry of International Application No. PCT/TR2023/051570, filed on Dec. 18, 2023, which is based upon and claims foreign priority to Turkey Patent Application No. 2022/019723, filed on Dec. 19, 2022, the entire contents of which are incorporated herein by reference.

### TECHNICAL FIELD

The invention relates to drug-loaded nanomotors prepared by electrochemical methods, which have applications in health sciences, pharmacy, pharmaceutical technology, nanotechnology, electrochemistry and pharmaceutical industry, and the use of these nanomotors for the treatment of Alzheimer's Disease.

### BACKGROUND

Alzheimer's Disease (AD), a sub-type of dementia, which is one of the central nervous system neurodegenerative diseases, is a disease whose incidence increases with the increase in the average age in developed societies. Treatment options include only symptomatic treatments and treatments to slow the progression of the disease despite this increase, and there is no treatment to prevent the causes of the disease.

The lack of effective treatment comes to the forefront as an unmet medical need even though approximately 40 million people are diagnosed with AD. It is estimated that the number of patients diagnosed with AD may reach 65.7 million in 2030 and 115.4 million in 2050 (Abbott, 2011, Alzheimer, 1907, Hebert et al., 2013, Prince et al., 2013, Scheltens et al., 2016, Selkoe, 2012). The costs of disease are expected to increase similarly to 1.1 trillion US Dollars in 2050 in parallel with this situation. It is necessary to develop new treatment approaches in the face of this situation, which will overshadow cancer and cardiovascular diseases, which are among the most common disease groups.

Many drug substances have been tried to be used in the treatment of Alzheimer's disease, but only 5 of them have been proven to be effective and accepted by international authorities. 4 of these substances are cholinesterase inhibitors and their names are Donepezil, Rivastigmine, Galantamine and Tacrine. Tacrine is a cholinesterase inhibitor that was first introduced in 1993 and has been discontinued due to its widespread side effects. Another molecule is memantine, which is an N-methyl-D-aspartate receptor antagonist. Molecules are FDA-approved drug substances indicated in Alzheimer's Disease. They are usually available on the market in tablet form and are used orally. However, rivastigmine is also available in patch form.

Conventional therapy forms are still applied today. However, conventional treatment methods cannot provide the expected and desired treatment in the treatment of Alzheimer's Disease, they only provide symptomatic treatment and slow the progression of the disease. New treatment approaches are needed due to their common side effects, the need for frequent dosing, the fact that they are not targeted to the site of effect, therefore, the transition of the drug to the domain is very low and the disadvantages such as patient compliance problems.

Nanosystems are very popular in new treatment approaches and give more effective results than conventional systems. Different drug delivery system applications of memantine HCl such as polymeric nanoparticle, nanosuspension, lipid-based nanosystem, chitosan nanogel are known in the art. However, new drug delivery systems studied to date are usually implemented through passive targeting or active targeting with surface modification. In addition, the observation of large nanoparticle sizes in some of these nanosystems makes it difficult for the drug to pass to the brain.

For these reasons, there is a need for drug delivery systems that can cross the blood brain barrier by directing with a magnetic field for the treatment of Alzheimer's disease without the need for surface function of the carrier nanosystem.

### SUMMARY

The present invention relates to nanomotors that meet the aforementioned needs, eliminate all disadvantages, and bring some additional advantages.

The primary object of the invention is to synthesize nanomotors (nanowires), which are poly-L-lysine polymers containing memantine HCl, aimed at the treatment of Alzheimer's Disease, and to provide the target to the brain by crossing the blood brain barrier by providing movement in the magnetic field.

The object of the invention is to synthesize memantine HCl-loaded nanomotors synthesized with a biocompatible poly-L-lysine polymer whose movement is directly controlled by the magnetic field and to provide its use in the treatment of Alzheimer's Disease. Nanomotors are loaded with memantine HCl synthesized with poly-L-lysine polymer. Memantine HCl-loaded nanomotors have both the superiority of movement and the superiority of targeting to the site of action and crossing the blood brain barrier with their controlled movement. Another solution brought by on-site treatment is to provide a therapeutic effect without the need for high doses applied in conventional treatment. Thus, common side effects observed in conventional treatments known in the art are reduced.

Another object of the invention is to provide the movement of the nanomotors to the desired region by directing with the direct magnetic field. There is no system such as a magnetic field that directly provides the movement of the nanosystem to the effect area in polymer-based nanosystems that were tried before nanomotors, and the surface of the nanosystem is functionalized and directed to the target. The travel to the target region (impact zone) is much more effective than other systems with the magnetic field used in nanomotors.

Another object of the invention is to synthesize nanomotors containing —COOH and —NH₂ functional groups on its surface. Thus, there is no need for any extra modification step to bind the drug substance.

### BRIEF DESCRIPTION OF THE DRAWINGS

Figures are provided for a better understanding of the nanomotor structure of the present invention:

FIG. 1 Optical microscope image of polymeric layer-based wire (rod) shaped motors

FIG. 2 SEM images of polymeric layer-based wire (rod) shaped motors.

FIG. 3 Schematic representation of polymeric layer-based wire (rod) shaped motors

### Reference Numbers

1: Polymer layer containing amino group
2: Pharmaceutical drug substance
3: Gold layer
4: Nickel plate

### DETAILED DESCRIPTION OF THE EMBODIMENTS

The invention relates to a polymer layer (1) containing the pharmaceutical drug substance (2) loaded amino group and a nanomotor containing a metal layer for orientation by magnetic field (FIG. 3). The invention uses nanomotors as carriers in the treatment of Alzheimer's disease.

The invention uses the NMDA antagonist drug as the pharmaceutical drug substance, the nickel metal layer (4) for magnetic field orientation and the poly-L-Lysine polymer layer and/or the PLGA-modified Poly-Lysine polymer layer as the polymer layer (1) containing the amino group.

The NMDA antagonist drug may be memantine or the pharmaceutical memantine salt memantine-HCl.

The poly-L-lysine polymer layer is located in the nanomotor basic structure. It is a biocompatible polymer and thanks to the activated surface, the drug substance memantine-HCl binding provided.

The nickel (Ni) layer is located in the nanomotor basic structure. It is the critical layer for orientation with magnetic fields.

The nanomotor preferably also comprises a gold (Au) layer (3). The gold layer has an influence on the morphological structure of nanomotors. It can also play a role in increasing the binding of the drug with different surface modifications. The gold layer has an influence on the shape of the motors. The rod shape of the structure is given properly, and the rod appearance of the motors is provided with the gold layer. The elongation of this layer causes the length of the motors to increase. It has been observed in the studies known in the art that a gold layer has been added to the structures twice. However, a smooth structure was provided without the need for a gold layer in the invention.

The bonded memantine HCl is the drug substance molecule attached to the nanomotor system thanks to the polymer layer in the basic structure of the nanomotors. It is the part of the drug substance that will provide the main treatment.

Memantine HCl-loaded poly-L-lysine polymer-based nanomotors (nanowires) were synthesized within the scope of the invention. The synthesized nanomotors are 200-300 nm in size. This polymer, in addition to being biocompatible, contains —COOH and —NH₂ functional groups in its structure and causes polymer-based platforms to be easily formed by using lysine amino acid as monomer by electrochemical polymerization. There is no need for any extra modification step and amino acid-based biocompatible nanomotors are not available in the literature for memantine transport and release since the synthesized motors contain functional surfaces. Metal-electroactive polymer preparation stages were carried out using the membrane template-assisted electrodeposition method in the synthesis of nanomotors. Electrochemical methods were used as the main method in nanofabrication. Electropolymerization and electrochemical coating methods have certain advantages in order to create homogeneous, stable, and robust structures. For this purpose, one side of the cylindrical alumina membranes was coated with silver (Ag) in a thin strip by sputtering method to serve as a working electrode and made conductive. Then, these coated membranes were placed in the teflon cell and polymerization, and metal deposition studies were carried out. Pt wire was used as the opposite electrode in the teflon electrochemical cell and Ag/AgCl electrode was used as the reference electrode. Poly-L-lysine was deposited on the surface in the presence of 10 mM L-lysine in a 50 mM (pH 7.4) phosphate buffer containing 0.1 M NaCl based on the amino acid monomer. Electropolymerization was carried out between −0.50 V and +1.80 V by alternating voltammetry method. At this stage, 3, 5, 10, 20 cycles of polymeric layers were formed. In addition, a polymeric layer was formed in constant voltage electrolysis (i-t) and a comparison was made.

This stage was performed at +1.6 V for 100 seconds. A load was passed through the system at constant voltage or current and gold (Au) and nickel (Ni) metals were deposited in the metal deposition stages. Copper (Cu), which acts as a sacrificing layer at −0.95 V, was metallically deposited from the copper sulfate (CuSO4) solution in the first stage. A load of 8 C was passed through the system at this time. Afterwards, Au deposition studies were carried out. A constant potential and current coating works were carried out in order to pass a constant load through the system at this time. The constant potential was kept at −1.0 V and the constant current was kept at −0.05 A. The Au solution used is the commercial coating solution (Orotemp, Italgalvano spa, Technic Group). Afterwards, Ni deposition studies were carried out. Ni deposition studies were carried out in the presence of nickel chloride (45 g/L NiCl₂·6H₂O) and nickel sulfate (300 g/L NiSO₄·6H₂O). Boric acid (45 g/L H₃BO₃) was also added at this stage because the pH value of the medium was important. Meanwhile, the load passing through the system was kept at a constant potential (at −0.95 V). The separation and washing processes of the nanomotors prepared electrochemically according to the membrane template-assisted electrodeposition method were carried out as follows: the Ag layer was carefully dissolved with alumina powder. Then, the sacrificial Cu layer was dissolved with 8 M HNO₃. The membrane was kept in 3 M NaOH solution for 1 hour to separate the nanowires from the mold and then washed with distilled water up to neutral pH after these processes. The precipitation steps were carried out at 6000 rpm (5 min) in the centrifuge in the washing stages.

Memantine HCl and CRANAD-2 dye loading stages were started after the synthesis of the basic structure of the nanomotors was completed. The activation approach with 1-ethyl-3-(3-dimethylaminopropyl)-carbodiimide/N-hydroxysuccinimide (EDC/NHS) was also used in the loading stage to ensure better interaction of the amino groups of memantine HCl and the carboxyl group of the polymer. Additionally, a coating study was conducted with Polysorbate-80. CRANAD-2 is a fluorogenic curcumin derivative that binds specifically to amyloid β plaques in the brain tissue of Alzheimer's patients and is used for near-infrared (NIR) detection and imaging in vivo. The detection and visualization of insoluble aggregates is crucial for assessing the therapeutic effect of anti-amyloid agents as well as for tracking disease progression. Optical microscopy methods are more suitable for the detection and examination of amyloids compared to nuclear or radiolabeled methods. CRANAD-2 is a commercially available curcumin derivative with a donor-recipient-donor architecture and is considered a promising amyloid fluorophore as it meets most of the probe requirements for amyloid detection and NIR imaging. CRANAD-2 recognizes insoluble amyloid plaques with nanomolar affinity, passes through the BBB, and shows a dramatic increase in NIR fluorescence upon binding to amyloid plaques. In particular, it shows fluorescence after binding to amyloid plaques and does not show any fluorescent radiation until it is connected.

At this stage, EDC/NHS treatment was performed for 15 minutes, 30 minutes and 60 minutes after the synthesis of poly-L-lysine-based nanomotors was performed. EDC/NHS solution was withdrawn from the motors that were centrifuged at 6000 rpm and precipitated to the bottom and the nanomotors were washed with water. 1 mL of distilled water was added to the collapsed nanomotors in the washing process and gently shaken and centrifuged at 6000 rpm for 5 minutes. A 5 mg/mL concentration of memantine HCl solution was added to the collapsed nanomotors in a volume of 1 mL. Incubation with this solution was performed for different periods of time, 60 minutes, 180 minutes and 1 day. Memantine HCl solution was then withdrawn from the nanomotors centrifuged at 6000 rpm for 5 minutes. Polysorbate 80 incubation was performed, and incubation was performed by adding 1% polysorbate 80 to the nanomotors in the same way. Finally, CRANAD-2 dye was incubated with the synthesized nanomotors and the paint was adhered to the surface. Incubations were also characterized voltammetrically during all these procedures.

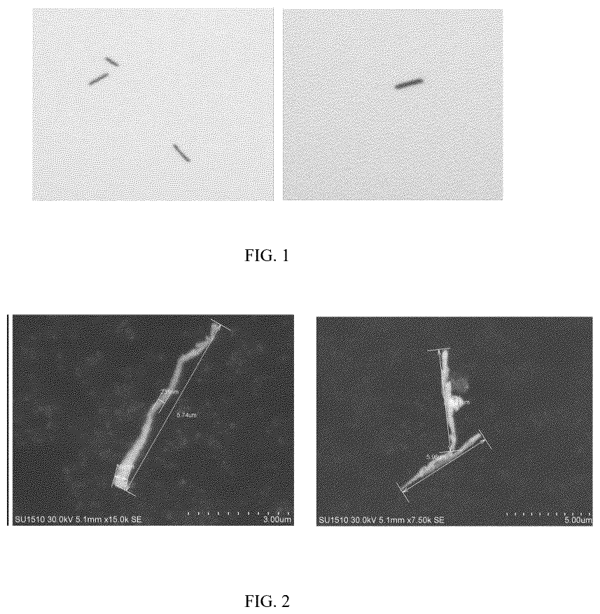

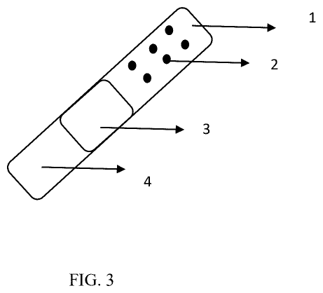

Nanomotors gain magnetic properties thanks to Ni deposition. Its movements are provided depending on its magnetic properties in the presence of an externally applied magnetic field. Nanomotors can be directed directly, so the system can be directed to the blood brain barrier. Whether the nanomotors that will reach the amyloid plaques in the brain, which is the area of action, adhere to the plaques can be observed with the fluorescent radiation that CRANAD-2 dye attaches to the plaques specifically. Microscope images of the prepared nanomotors are shown in FIGS. 1 and 2.

Nanomotors are synthesized according to the membrane template-assisted electrodeposition method and the pores of these membranes used as molds are 200 nm in diameter. For this reason, the diameter of the motors is also expected to be 200 nm. There may be some expansion when separating from the mold. The diameters of the obtained motors are also in the range of 200-300 nm. Their lengths were found to be in the range of 3-5 micrometers. Their size should be around 200 nm to overcome the blood brain barrier.

1. A nanomotor comprising the following:
a polymer layer containing an amino group loaded with a pharmaceutical drug substance,
a metal layer for orientation with a magnetic field.

2. The nanomotor according to claim 1, wherein the polymer layer containing an amino group is a poly-Lysine polymer layer and/or a PLGA-modified poly-Lysine polymer layer.

3. The nanomotor according to claim 1, wherein the metal layer is a nickel metal layer.

4. The nanomotor according to claim 1, wherein the pharmaceutical drug substance is a NMDA antagonist drug.

5. The nanomotor according to claim 4, wherein the NMDA antagonist drug is memantine or Memantine HCl, the pharmaceutical Memantine salt.

6. The nanomotor according to claim 1, further comprising gold layer.

7. The nanomotor according to claim 1, wherein the nanomotor is 200-300 nm in diameter.

8. A method of using the nanomotor according to claim 1 for treatment of Alzheimer's Disease.

9. The method of using the nanomotor according to claim 1 for treatment of Alzheimer's Disease, wherein the polymer layer containing an amino group is a poly-Lysine polymer layer and/or a PLGA-modified poly-Lysine polymer layer.

10. The method of using the nanomotor according to claim 1 for treatment of Alzheimer's Disease, wherein the metal layer is a nickel metal layer.

11. The method of using the nanomotor according to claim 1 for treatment of Alzheimer's Disease, wherein the pharmaceutical drug substance is a NMDA antagonist drug.

12. The method of using the nanomotor according to claim 1 for treatment of Alzheimer's Disease, wherein the NMDA antagonist drug is memantine or Memantine HCl, the pharmaceutical Memantine salt.

13. The method of using the nanomotor according to claim 1 for treatment of Alzheimer's Disease, further comprising a gold layer.

14. The method of using the nanomotor according to claim 1 for treatment of Alzheimer's Disease, wherein the nanomotor is 200-300 nm in diameter.
